(19) United States
(12) Patent Application Publication
(10) Pub. No.: US 2026/0020962 A1
(43) Pub. Date: Jan. 22, 2026

(54) FLUID EXCHANGING ELECTRODE AND RELATED SYSTEM

(71) Applicant: Garwood Medical Devices, LLC, Buffalo, NY (US)

(72) Inventors: Jackson G. Hobble, Tonawanda, NY (US); Timothy M. Kopera, Lancaster, NY (US); Morgan Schnee, Buffalo, NY (US); Brian R. Peterson, East Aurora, NY (US)

(73) Assignee: Garwood Medical Devices, LLC, Buffalo, NY (US)

(21) Appl. No.: 18/867,337

(22) PCT Filed: May 16, 2023
(86) PCT No.: PCT/US2023/022344

§ 371 Date: Nov. 19, 2024

Related U.S. Application Data
(60) Provisional application No. 63/343,651, filed on May 19, 2022.

(51) Int. Cl. A61F 2/30; C25F 1/00

(52) U.S. Cl. CPC A61F 2/30; C25F 1/00; A61F 2002/30668; A61F 2002/30719; A61F 2310/00011

(57) ABSTRACT

A fluid exchange electrode is defined by a multi-layered electrode body including a conductive electrode material layer disposed in an electrode body and an ionically conducting assembly that interfaces with the skin of a patient. A source of electrolytic fluid is coupled to an inflow tube and configured to permit a volume of electrolytic fluid to be circulated through the electrode body to increase treatment duration and overall electrode life in which the temperature of the cycled electrolytic fluid can be regulated. The cycled electrolytic fluid can be disposed of or preferably recirculated via a return or outflow tube to the source of electrolytic fluid. The herein described fluid exchange electrode can be used as part of a treatment system that applies a DC voltage in order to remove bacteria from a metal surgical implant.

### CROSS REFERENCE TO RELATED APPLICATION

This application claims priority to U.S. Application Ser. No. 63/343,651, filed May 19, 2022, and entitled: Fluid Exchanging Electrode and Related System, which is herein incorporated by reference in its entirety.

### TECHNICAL FIELD

This application is directed generally to the field of surgically or orally implantable devices and more specifically to a novel design for an electrode, as well as a treatment system employing the electrode. In one version, the electrode/treatment system is used for providing DC voltage stimulation to enable the removal of biofilms and bacteria from metallic surfaces of an implant. According to at least one embodiment, the electrode cycles electrolytic fluid from an external reservoir to enable longer safe treatment durations, as well as an expanded lifespan of the electrode.

### STATE OF THE ART

Metal implants are used in patients with many different injuries or medical issues. More specifically, metal implants may be used for any individual that needs to replace one of more joints. According to one example, an orthopedic metal implant may be used to replace a patient's hip or knee. According to yet another example, a dental implant may be used to replace a patient's tooth. A potential problem with metal implants is that they tend to allow for the growth of bacteria on the metallic surface of the implant over time. This may increase the patient's risk for an infection with the patient facing the risk of having additional surgery in order to remove and replace the implant. To decrease the risk of infection, electrodes can provide electrical stimulation to an existing implant that disrupts the growth of bacteria. Systems have been developed in which a sufficient cathodic current can be subdermally applied to metal implants in order to create electrochemical reactions at the metallic surface of the implant that can disrupt and kill formed bacterial biofilms.

For electrochemical processes to occur, there must be at least two (2) electrodes, namely an anode and a cathode within an electrolyte solution. The anode is a metallic surface at which oxidative reactions occur, with the cathode being another metallic surface at which reduction reactions occur. A reduction reaction is essentially when a material of interest gains electrons and thereby decreases the oxidation state of the molecules. The electrolyte in which the anode and cathode each reside in provides an electrical connection by facilitating the flow of electrons shuttled by ion carriers, such as sodium or potassium ions. Electrons are driven from the anode to the cathode along an electrical path via a potentiostat. A potentiostat is an instrument configured to drive current from a counter electrode to a working electrode in order to keep the voltage on the working electrode at a constant value, and more preferably as compared to a stable reference electrode of the treatment system. In the case of Cathodic Voltage Controlled Electrical Stimulation (or CVCES), the anode represents the counter electrode, and the cathode represents the working electrode. Using a potentiostat, a user can dictate which electrochemical process is occurring on the working electrode and at what rate the process occurs simply by adjusting the applied voltage parameters. The counter electrode has specific physical, electrical, and chemical requirements, which must be met in order to sufficiently facilitate CVCES treatment, especially in a clinical environment in which a patient's health is concerned.

The CVCES treatment technique in a clinical setting has been shown as an effective way to fight bacterial biofilm infections on metallic implants in a very minimally invasive manner without surgical intervention. In this setting, the patient's body acts as an electrochemical cell by using the metal implant as the working electrode (cathode), with the counter electrode attached to the skin surface of the patient acting as the anode.

A counter electrode previously designed by Applicant is described in WO 2021/178040A1, and entitled: Circumferential skin electrode for use with metal surgical implants. This design relates to a counter electrode that can be used in a CVCES-based treatment system. A circumferentially disposed electrode design has been found to increase the efficiency of treatment while maintaining patient safety parameters, as well as present a minimally invasive profile. The general components of this known electrode design includes a lead wire, a conductive mesh layer, a conductive anodic film layer, a buffered hydrogel layer, and a preferred geometry. A significant feature of this design is that the electrode is shaped and configured to be fitted around substantially most of or preferably the full circumference of the patient's limb. This preferred geometry increases the therapy distribution on the implant and also increases the lifespan of the counter electrode. In prior treatment systems, the counter electrode was a common patch that was locally disposed on only one side of the implant. As a result, the natural tendency of the electrochemical reaction would become more intense on the side of the implant closest to the electrode, thereby creating an uneven treatment on the implant. This uneven treatment happens because applied current will naturally flow through the path of least resistance from the implant to the counter electrode. The distance between the counter electrode and the opposite side of the implant increases the natural resistance between the two electrode zones and thus less current will flow there. There is a correlation between anode-to-cathode distance and resistance. Resistance will also change depending on other factors in the treatment system such as muscle composition, fat composition, bone, skin hydration, and overall body hydration.

Ultimately, this uneven treatment causes the side of the implant with high densities of cathodic reactions to receive a disproportionate amount of treatment as compared to the low-density reactive side of the implant, thereby creating a clinical treatment which is far from ideal. Accordingly and using a circumferentially disposed electrode as described by Applicant in WO2021/178040A1, these resistance factors can be normalized to a higher degree than traditional patch electrodes. More specifically, providing treatment using a circumferentially disposed electrode provides a more consistent and predictable treatment to disrupt any bacterial biofilm found on the implant. Consistent treatment is especially important because if portions of the biofilm do not receive adequate treatment, the biofilm is liable to simply regrow and continue to cause infectious problems for the patient.

A second improvement that the circumferential design described in WO2021/178040A1 demonstrates over traditional patch electrodes is improved safety for the patient's skin and tissue that surrounds the implant. Depending on the amount of current entering the body, concentrating all of the current locally to one spot could cause severe chemical burns or thermal necrosis to the localized tissue, especially the skin to which the patch electrode is adhered. Specifically, the counter electrode creates a buildup of acidic byproducts, and as the electrochemical reactions proceed over time, the acid will become more concentrated. If a low (i.e., acidic) pH is created and in contact with the skin for extended periods of time, the skin will experience chemical burns. The above-described circumferential electrode helps mitigate this risk through its large surface area, which allows the current entering the body to become much more widely distributed over the surface of the electrode/skin. This latter feature subsequently lowers the anodic current density, slows down the decrease in pH per unit of area, and creates a safer treatment for the patient.

While the features of the counter electrode described in Applicant's WO2021/178040A1 are quite beneficial for promoting a safe treatment to infected implants, it has been found that limitations may still occur within longer therapies; that is, treatments lasting 3 hours or more. Extended treatment durations cause excessive buildup of acidic byproducts within the hydrogel layer that interfaces the conductive electrode surface with the skin, and simply overcomes the electrode's ability to distribute current over its large size. While the prior circumferential design describes a buffering system within its hydrogel that combats acidic byproducts, this feature also has limitations especially over an extended treatment duration. Additionally, it has been observed that at high treatment levels, this form of electrode provides inadequate thermal regulation of heat generation at the patient's skin. Accordingly, there remains a need in the field for an electrode design that resolves the fundamental issue of acidic byproduct production during the timespan of DC stimulation application, such as those in CVCES treatments, while also providing a means more effective thermal regulation at the application site of the electrode.

### BRIEF DESCRIPTION

The herein disclosed invention presents a novel way of significantly improving the treatment lifespan of the counter electrode component of an implant treatment system while also maintaining key features of electrodes deemed critical for patient safety and treatment efficacy.

Therefore and according to at least one aspect of the present invention, there is provided a system for treatment of metal implants for the removal of bacteria, the system comprising a device capable of producing a DC voltage, a working electrode coupled to the device capable of producing a DC voltage, the working electrode being the metal implant, and a counter electrode coupled to the device capable of producing a DC voltage. The counter electrode comprises a multi-layered electrode body having a conductive electrode material layer disposed in a flow chamber of the electrode, and an ionically conducting assembly configured to directly contact the skin of a patient. A source of electrolytic fluid is coupled to the counter electrode and configured to permit a volume of electrolytic fluid to flow through the electrode body.

According to at least another aspect, there is provided a fluid exchange electrode comprising an electrode body, a conductive electrode material layer disposed within the electrode body; an ionically conductive assembly configured to interface with a skin surface of a patient; and an inflow tube and an outflow tube coupled to an interior of the electrode body, wherein the inflow tube is configured for connection to a source of electrolytic fluid that permits a volume of electrolytic fluid to flow through the electrode body.

According to yet another aspect of the invention, there is a method for increasing the lifespan of an electrode used for biofilm removal treatment of implants, the method comprising: coupling a source of electrolytic fluid to the electrode; and causing electrolyte fluid to be circulated into the electrode during treatment wherein the electrode includes an electrode body having an interior flow chamber disposed between a pair of insulation layers, and an electrically conductive electrode surface layer disposed within the defined flow chamber, the method further comprising the steps of providing a plurality of conduction perforations in one of the insulation layers, and providing an ionically conducting assembly beneath the insulation layer having the plurality of perforations and a skin surface of a patient.

In brief, the present invention is based on a treatment system in which a DC electrical current is applied to a metal implant, such as a knee or hip replacement, in order to electrochemically clear and disrupt harmful bacterial biofilm from the metallic surface of the implant. The treatment system requires at least two (2) electrodes to effectively transfer the DC electrical current from a voltage supply to the metal implant. One of the electrodes (a working electrode) is the metal implant itself, which is connected to the voltage supply by subdermal attachment. The second electrode, which is referred to as the counter electrode or return electrode, is adhered directly to the skin of the patient in the vicinity of the implant. As the treatment provides a DC current from one electrode to another, acidic byproducts will generate on the counter electrode stimulating surface via transduction of electrons into the surrounding electrolyte. Treatment lifespan of the counter electrode has traditionally been limited by the acidic buildup in the hydrogel to skin interface of the electrode, which can eventually cause chemical burns on the patient's skin.

The disclosed invention provides a novel approach to continually clear acidic byproducts out of the electrode via cycling of an electrolytic fluid. This fluid cycling significantly improves the lifespan of the electrode by preventing the patient's skin to interact with acidic byproducts. The fluid cycling additionally provides thermal regulation to the skin site, which otherwise has been observed to rise in temperature to undesirable levels in the absence of the electrolytic fluid. According to at least one embodiment, the counter electrode is defined by a multi-layered structure that is configured to permit a conductive electrolyte to flow into and out of the electrode body, thereby transporting acidic byproducts out of the electrode and fresh media (fluid) into the electrode. Electrical conduction to the implant is not affected by this design and key performance parameters are not sacrificed.

A number of challenges were encountered in developing a suitable electrode and system design. These challenges, which are also discussed in greater detail in the Detailed Description, include the following:

One challenge encountered by Applicants was in creating a way to isolate the skin-interfacing hydrogel from the internal electrolyte flow without blocking electrical conduction and not allowing water/high moisture content material to directly contact skin. As is known, hydrogels swell massively when in contact with water, which can negatively affect the mechanical integrity and adhesiveness of the hydrogel. Accordingly, a material was needed that could allow for electrical conduction through its body, prevent water from contacting the hydrogel, and not create its own redox reaction.

Another challenge encountered was in optimizing the treatment or electrode system such that excessive amounts of electrolytic fluid exchange were not needed in order to achieve an increased lifespan of the electrode. This challenge was resolved by incorporating additives in the electrolytic fluid, both in the initial volume and periodically later in treatment, to enable the electrode to achieve lifespan milestones by recycling the same electrolytic fluid. Total volume could therefore be significantly smaller than initial prototypes.

In addition, the upper and lower insulation layers of the electrode defining a flow chamber should be fluidically sealed in a leak proof manner. One potential problem area in maintaining this seal is that of the extending wire linked to the potentiostat or other current delivering device, and more specifically where the extending wire protrudes through the electrode body. To overcome this challenge, a heat source with a tip can be used to ensure the insulation layers are fused together in proximity to the extending wire.

Still further, and with the insulation layers each being preferably made from a thin flexible material there was a challenge in the layers forming the enclosed flow chamber in the electrode body being pinched together, and potentially preventing the cycled electrolytic fluid from flowing freely. To solve this issue, a woven mesh sheet or layer was added in between the insulation layers to add stability and support and to prevent the flow of electrolytic fluid in the electrode from otherwise being obstructed.

The present design significantly improves the lifespan of a return or counter electrode in treatment systems that provide DC stimulation to metallic implants. Previous art used to provide electrical stimulation to metallic implants are limited in their duration or lifespan due to the buildup of acidic byproducts in their hydrogel to skin interface, creating the risk of chemical burns to the patient. The disclosed invention addresses this issue while also maintaining the benefits of existing electrodes used for biofilm removal/eradication, including evenly distributed therapy on the metallic surface of the implant itself as well as a number of safety-specific features used to preserve skin health.

One of the reasons that this disclosed design is unique is because the herein described electrode uses materials and features that are completely foreign to the world of standard medical electrodes. That being said, this novel design fills a large need in the field and particularly to treatment systems reliant on DC stimulation for eradication of orthopedic and other implant infections.

Advantageously, the herein described electrode is configured to permit treatments of much longer duration than known electrodes used for the same or similar purposes, but without impacting performance. In addition, patient safety is maintained if not improved while the overall lifetime of the electrode is increased.

These and other features and advantages will be readily apparent from the following Detailed Description, which should be read in conjunction with the accompanying drawings.

### BRIEF DESCRIPTION OF THE DRAWINGS

The accompanying drawings, which are incorporated herein and constitute part of this specification, illustrate presently preferred embodiments of the invention, and together with the general description given above and the detailed description given below, serve to explain features of the invention (in which like reference numerals represent like elements or steps) of which:

FIG. 1 is an exploded view of a fluid exchange electrode that is made in accordance with aspects of the present invention;

FIG. 2 is a partial side elevational view showing the application of electrolytic fluid to the electrode of FIG. 1 in accordance with aspects of the present invention;

FIG. 3(a) is a schematic view of a treatment system incorporating a fluid exchange electrode according to aspects of the present invention;

FIG. 3(b) is a schematic view of another treatment system incorporating a fluid exchange electrode in accordance with other aspects of the present invention;

FIG. 4(a) is an exploded view of another fluid exchange electrode made in accordance with aspects of the present invention and more specifically a fluid exchange electrode having a dual channel configuration;

FIG. 4(b) is a top perspective view of the fluid exchange electrode of FIG. 4(a);

FIGS. 5(a) and 5(b) are comparative top perspective views of electrodes made in accordance with aspects of the present invention; and

FIG. 6 represents a graphical representation of test data depicting pH change over time for various prototype electrodes made in accordance with the present invention.

### DETAILED DESCRIPTION

The following description relates to a number of specific embodiments of a novel electrode design made in accordance with aspects of the present invention. More specifically, the described embodiments are each specific to a counter or return electrode configured for use in a CVCES-based treatment system for the eradication or disruption of bacterial films from surgical metallic implants. It will be apparent from the following description, however, that the herein described electrode design can be configured for use in other suitable applications. In addition and throughout this detailed description, several terms are used in order to provide an adequate frame of reference in regard to the accompanying drawings. These terms, which may include “front”, “rear”, “back”, “end”, “proximal”, “distal”, “top”, “bottom” and the like are not intended to significantly limit or otherwise affect the intended scope of the invention, except where so specifically indicated.

The accompanying drawings are merely intended to illustrate and present salient features of the present invention. Accordingly, the drawings should not be relied upon by the reader for scaling or similar purposes. As to specific dimensions used for components throughout the following description, the range is intended to be ±15 percent; that is, 1.0 inch represents a range of 0.85-1.15 inches.

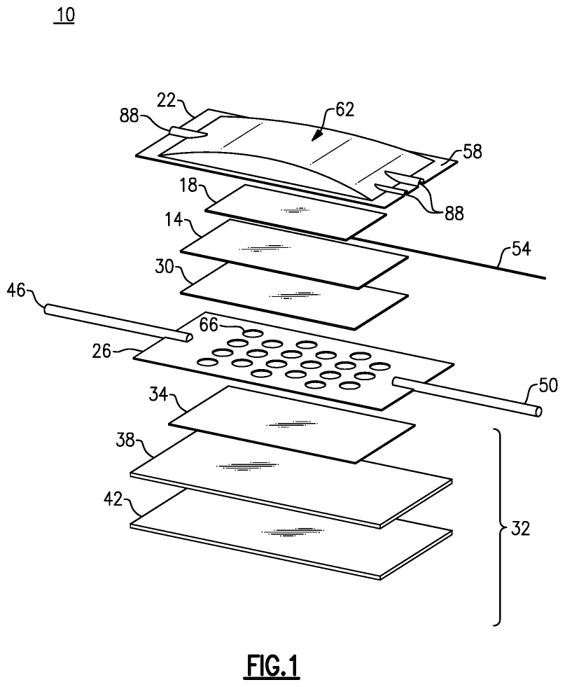

With reference to FIG. 1, there is shown a fluid exchange electrode 10 made in accordance with a first embodiment of the present invention, and more specifically a counter or return electrode for use in a CVCES treatment system. The fluid exchange electrode 10 is defined by a plurality of layers that combine to form a stacked structure. More specifically, the counter electrode 10 according to this specific embodiment includes a conducting electrode surface layer 14, an optional metallic backing layer 18, an insulative backing 22 that seals to an insulating layer 26, a mechanical support mesh layer 30, an ionically conductive membrane layer 34, and at least one hydrogel layer. In this specifically depicted embodiment, a pair of hydrogel layers, namely an inner hydrogel layer 38, and an outer hydrogel layer 42 are provided, in which the outer hydrogel layer 42 is configured to interface with the skin of a patient. Also and as further discussed herein, the ionically conductive membrane layer 34 and the at least one hydrogel layer (e.g., layers 38, 42) form an ionically conductive assembly 32 of the herein described electrode 10, which is disposed beneath the insulation layer 26.

In addition, the counter electrode 10 according to this specific embodiment includes a number of other components connected or otherwise coupled to the body of the herein described electrode 10. These components include an electrical lead 54 extending from the optional metallic backing layer 14 to a potentiostat or other similar device (not shown in FIG. 1) that is capable of generating a sufficient DC voltage for CVCES-based implant treatment, as well as an inflow tube 46, and an outflow tube 50 that are configured to circulate or cycle an electrolytic fluid (also synonymously referred to throughout this description as “electrolyte” or an “electrolyte fluid”) through defined or formed portions of the electrode 10. Each of these various components of the electrode 10 will now be described in greater detail.

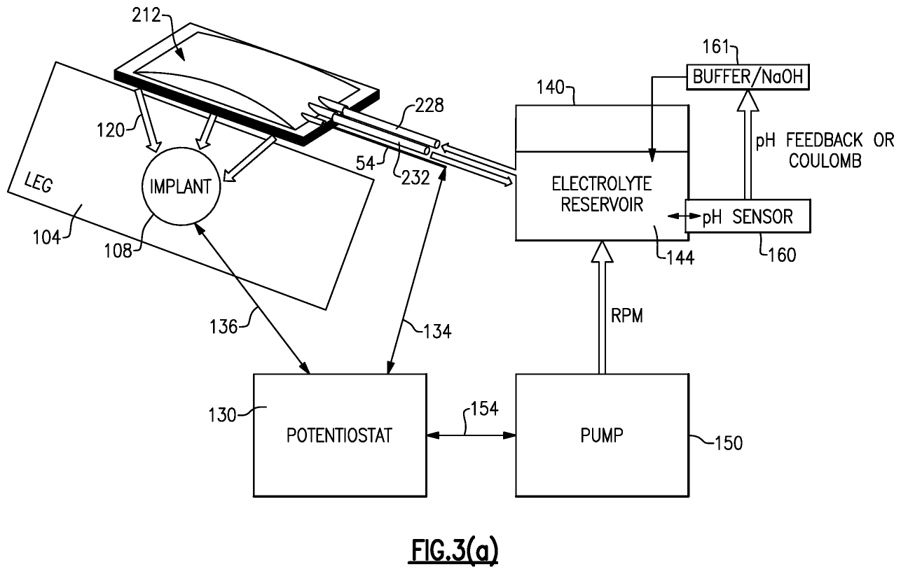

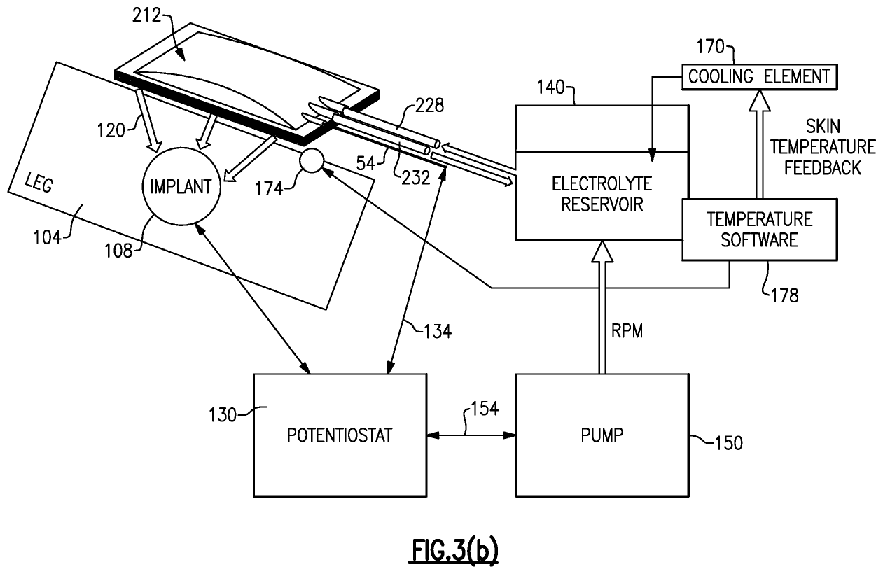

A foundational component to any counter electrode design is the conducting electrode surface layer 14, which is intended to conduct electrical energy to the patient's body and the metal implant within, therefore representing the anode of a CVCES-based treatment system. The conducting electrode surface layer 14 can be made from any inert, electrically conductive material that does not degrade or produce its own chemical byproducts when performing as the anode of the treatment system. According to the herein described embodiment, the conducting electrode surface layer 14 is made from a carbon vinyl sheet. Carbon vinyl is a common substrate material that can be used in stimulating electrodes largely due to its overall flexibility, which can easily conform to the skin of a patient. Other suitable materials that can also be utilized for this purpose include chemically inert metals, which may include, for example, platinum and rhodium, among others. These alternative metals may be provided either as a mesh (e.g., a grid of wires), or as a thin sheet. As discussed in greater detail below, the conducting electrode surface layer 14 connects electrical flow from a potentiostat, such as schematically shown as 130 in FIGS. 3(a) and 3(b), or other power source capable of providing sufficient electrical energy via the electrical lead 54, which can be adhered or epoxied to the surface of the conducting electrode surface layer 14 or the optional metallic backing layer 18.

The optional metallic backing layer 18 preferably laminates the upper facing side of the conductive electrode surface layer 14. This layer 18 may be made from a conductive metal foil or mesh which may include copper or platinum, or alternatively the metallic backing layer 18 can utilize a conductive ink. The metallic backing layer 30 is designed to help distribute current generated from the potentiostat or similar device over the entirety of the conductive electrode surface layer 14, since carbon vinyl is typically not sufficiently conductive to evenly distribute the electrical current. Therefore if the conductive electrode surface layer 14 is not formed from a carbon vinyl sheet, but rather is formed from another metal having sufficient conductivity such as platinum or rhodium, then the metallic backing layer 18 becomes an optional component of the electrode 10. Ideally and according to this embodiment, the metallic backing layer 18 has a smaller surface area or footprint than that of the conductive electrode surface layer 14. More specifically, the edges of the metallic backing layer 18 are slightly recessed from the edges of the conductive electrode surface layer 14 to prevent contact with electrolyte fluid, which would create an undesirable interaction in the electrochemical reaction.

To provide sufficient electrical insulation from the conductive electrode surface layer 14 for the patient or caregiver, the insulative backing 22 is applied over the conducting electrode surface layer 14 and the metallic backing layer 18, thereby effectively sealing the metallic backing layer 18 between the conductive electrode surface layer 14 and the insulative backing 22. The electrical lead 54 is adhered or otherwise attached to the metallic backing layer 18, as shown in FIG. 1. In the instance in which there is no metallic backing layer 18, then the electrical lead 54 would be attached to and extend outwardly from the conductive electrode surface layer 14.

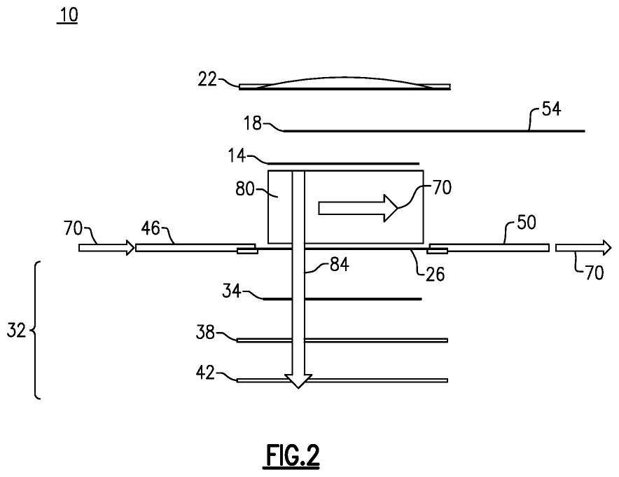

As further depicted in FIG. 1, the insulative backing 22 may seal to the insulation layer 26 via an edge or peripheral seal shown as 58, wherein the insulative backing 22 preferably acts as a cover of the herein described electrode 10. Moreover, the insulative backing 22 includes a domed or concave surface that creates a consistent cross-sectional height of a defined interior chamber having an open volume 62 enclosing both the conducting electrode surface layer 14 and the metallic backing layer 18, which are both sealed to the inside ceiling of the defined interior chamber. The edge seals 58 enable the electrode 10 to be leak proof and easily assembled during manufacturing. Accordingly, an enclosed interior volume is provided through which electrolytic fluid 80, FIG. 2, will flow transversely and cycle through the electrode body via the inflow tube 46 and the outflow tube 50, each of which are hermetically sealed between the insulative backing 22 and the insulation layer 26 by means of the peripheral seal 58. The insulative backing 22 and the insulation layer 26 together may be composed of a suitable plastic, such as polyvinyl chloride or PVC. Ports 88 formed in the edge seals 58 as indentations in respective opposing ends of the insulative backing 22 allow for connection to external flow tubing that will interact with a pump 150, such as shown in FIGS. 3(a) and 3(b), and an electrolyte reservoir 140, also shown in FIGS. 3(a) and 3(b). More specifically, the external flow tubing is the inflow tube 46, the outflow tube 50 and the electrical lead 54.

According to this specific embodiment, the insulation layer 26 includes a pattern of spaced conduction through perforations 66 formed along the surface of the insulation layer 26 opposite the conducting surface. The perforations 66 according to this specific embodiment are defined by an evenly spaced set of circular openings, but it will be understood that the perforations may assume other convenient shapes including but not limited to elliptical, rhombic, and polygonal cross sections. This latter feature creates one or more open areas that are sufficiently sized to enable ionic electrical conduction through the encapsulated electrolytic fluid 80, FIG. 2, to the adjacent layers of the formed fluid exchange electrode 10. In this specific embodiment, the diameter of the conduction holes 66 are 0.5″, but can range between about 0.01″-5.0″. The spacing between each of the conduction holes 66 is approximately 0.75″ center to center according to this specific embodiment, but this parameter can range from about 0.01″ to 5.0″.

Also disposed between the conducting electrode surface layer 14 and the insulating layer 26 is the mechanical support mesh layer 30 that prevents pinching of the formed flow chamber defined by the interior (open) volume 62. Preferably, the mechanical support mesh layer 30 is made from a moisture-resistant plastic mesh of plastic wire strands assembled thereto in a woven grid pattern. Alternatively, the support mesh layer 30 could be formed by injection molding. In this specific embodiment, the diameter of these wire strands are 0.05″, but the diameter of the plastic wire strands can range between 0.001″-0.25″. Disposed beneath the insulation layer 26 is the ionically conducting assembly 32, which according to this embodiment is made up of a specialized ionically conductive membrane layer 34 and the inner and outer hydrogel layers 38, 42, each of which will be described in greater detail below.

FIG. 2 represents how electrolytic fluid is caused to flow through the fluid exchange electrode 10 of FIG. 1. More specifically, a volume of electrolyte fluid, shown schematically as 80, is provided between the conducting electrode surface layer 14 and the insulation layer 26. The composition of the electrolytic fluid will be discussed in depth, but on a cursory level the electrolytic fluid is composed of water, a conduction metallic salt, and a neutralizing agent each being mixed in solution. The neutralizing agent may include a hydroxide salt, a buffer, or a combination of each. A viscosity increasing agent may also be present in the solution, enabling the electrolyte fluid to be a viscous gel. The directional flow of the electrolyte fluid 80 is depicted functionally by arrows 70 wherein the electrolytic fluid 80 enters the electrode 10 transversely through the inflow tube 46 and exits the electrode 10 via the outflow tube 50. Ionic transduction of energy is functionally depicted by downwardly pointing arrow 84 in which the transduction occurs from the conducting electrode surface layer 14, through the encapsulated electrolytic fluid 80, and further through the ionically conducting assembly 32, which according to this specific embodiment, is made up of the ionically conductive membrane layer 34, and hydrogel layers 38, 42, respectively, and into the body of the patient (not shown) to interact with an infected metal implant (also not shown in this view). This electrical flow is driven by the external potentiostat, galvanostat, or power supply (not shown) via the coupled electrical lead 54. As described herein the inflow tube 46 and the outflow tube 50 can be coupled through appropriate tubing to an electrolytic reservoir such as 130, FIGS. 3(a) and 3(b), to create a fluid circuit relative to the electrode body or alternatively, the outflow tube 50 can be connected to a waste receptacle (not shown).

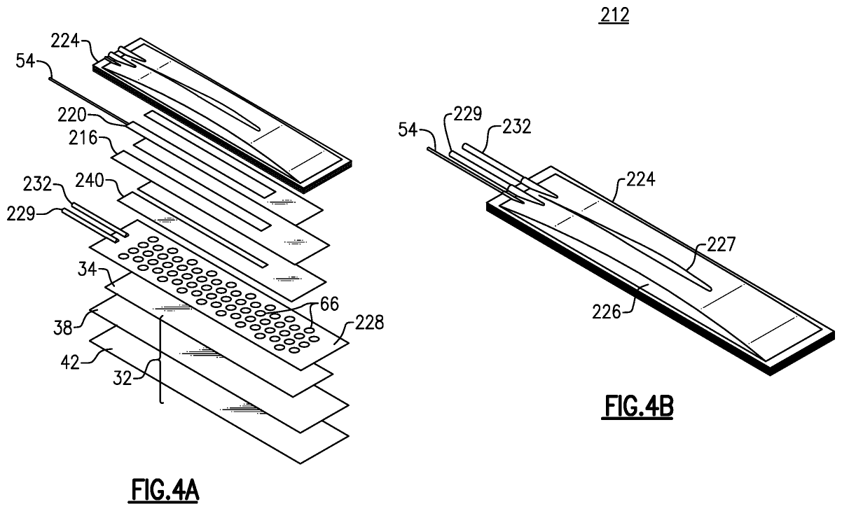

Other suitable variations and modifications can also be considered for the electrode design. For example, another alternative configuration for a counter or return electrode design is the incorporation of dual parallel channels as shown in FIGS. 4(a) and 4(b). In briefly describing this latter embodiment, it should be noted that similar components are labeled with the same reference numbers for the sake of convenience. As in the case of the electrode 10, the fluid exchange electrode 212 according to this embodiment is made up of a plurality of components formed in a stacked structure. More specifically, the electrode 212 includes a conductive electrode surface layer 216, an insulating layer 228 having a pattern of spaced conduction perforations 66, and an insulative backing 224 which forms a cover for the electrode 212 with the insulating layer 228 and the insulative backing 224 combining to form an interior flow chamber. The herein described electrode 212 further includes an optional metallic backing layer 220 and a mesh or support layer 240, each disposed within a sealed interior volume 226 of the formed flow chamber, as well as an ionically conducting assembly 32 made up according to this embodiment by an ionically conductive membrane layer 34 and inner and outer hydrogel layers 38, 42. Like the previously described design, the ionically conducting assembly 32 is disposed beneath the insulating layer 228.

A distinction as compared to the prior fluid exchange electrode design 10 of FIG. 1, is that the inflow and outflow tubes 229, 232 are disposed on the same side of the electrode body. This latter configuration permits two (2) parallel channels to be formed within the defined interior volume 226, ensuring a path for electrolytic fluid to circulate through the electrode 212. Additonally, the mechanical support mesh layer 240, the conducting electrode surface layer 216 and the optional metallic backing layer 220 according to this embodiment have each been modified to assume a substantial U-shape with the opening towards the parallel inflow and outflow tubes 229, 232. An additional sealing location is provided in the opening by an indented portion 227 of the insulative backing 224 between the channels of each of the U-shaped layers, in which the Insulative backing 224 is sealed to the insulating layer 228. This additional seal created by the indented portion 227 creates the defined parallel channels as part of the open internal volume 226 within the fluid exchange electrode 212.

Aspects of the ionically conducting assembly 32 disposed below the insulation layer 26, 228 for each of the foregoing electrode designs are now discussed. One challenge encountered by Applicants during the development of this invention was maintaining the integrity of the skin-interfacing outer hydrogel layer 42, especially when aqueous electrolyte fluid 80 is in direct contact therewith. Hydrogels are typically solid gel materials that are composed of a fibrous mesh, water, and conductive salts. These materials are commonly used to interface the sensor or stimulating surface of the electrode to the skin. It is a known behavior of hydrogels to swell immensely when in contact with aqueous solutions, therefore compromising their mechanical durability and adhesiveness. An original design intent of the invention was to adhere a hydrogel layer directly to the outside of the insulation layer having the spaced conduction perforations 66, which could then adhere to the skin of a patient. Conduction could then progress from the conductive electrode material layer within the internal volume of the electrode, through the electrolytic fluid 80 and conduction perforations 66, and finally through the hydrogel layer(s) to the skin of the patient. Naturally, when the internal volume of the electrode was filled with electrolyte that interfaced with a hydrogel, the hydrogel swelled within a short period of time to a level that compromised electrode usability.

Accordingly, a need was perceived for the additional ionically conductive membrane layer 34, which is disposed between the inner hydrogel layer 38 and the insulation layer 26 (direct contact through the conduction perforations 66, FIG. 1) that would allow for ionic conduction, while also limiting water transport. By “ionic conduction”, it is meant that the ionically conductive membrane layer 34 cannot be formed from a piece of metal, for example, because metal would require that the electrons be transported electrically instead of ionically, which would cause redox reactions on each side of the metal and therefore defeat the purpose of an ionically conductive membrane layer as acid would then build up in the hydrogel. In this embodiment, the membrane layer 34 has a volume resistivity of less than 1,000 ohm-cm, but the ionically conductive membrane layer 34 may have a volume resistivity that is less than 1,000,000 ohm-cm. In the herein described embodiment, the thickness of the ionically conductive membrane layer 34 is between 100 and 200 micrometers, but it will be understood that the thickness of this layer 34 may fall anywhere in the range between 1 micrometer and 1 centimeter. As now described, several classes of materials were evaluated and deemed suitable for the application, but among available options, certain classes of materials have different properties that lend to subsequent deficiencies in performance, usability, or manufacturability. More specifically, it has been determined that the following classes, of materials, namely solid state electrolytes, anion exchange membranes and cation exchange membranes can be considered.

Solid state electrolytes include NASICONs or LISICONs,. Anion and cation exchange membranes, commonly used in chlor-alkali production cells, are further options for the ionically conductive membrane layer 34. Anion exchange membranes are semipermeable membranes generally made from ionomers and designed to conduct anions, such as hydroxide ions, but reject gases such as oxygen or hydrogen, as well as cations. Cation exchange membranes allow the transport of cations, such as protons and sodium ions, while blocking the conduction of anions, such as hydroxides. This transport allows for conductivity to be higher as compared to anion transport membranes, providing minimal extra voltage requirements on a CVCES-based or other treatment stimulating device. Cation exchange membrane materials can very readily accept a free electron, making this material electrically conductive. Therefore, when this cation exchange material is laminated to a hydrogel, the water in the hydrogel, which is comprised of H+ ions and —OH groups, is separated as the cation exchange membrane absorbs the —OH groups and leaves excess H+ in the water, causing the pH of the hydrogel to decrease. Also, as a part of its conduction mechanism, when the cation exchange membrane material is exposed to water, the material self-organizes nanocavities within which water can diffuse. The diffusion of water facilitates at least a portion of protonic transport that is a key element of its ionic conductivity. Because water diffusion will affect the mechanical and adhesive properties of the laminated hydrogel, a number of cation exchange membranes preferably create a limit on how long the counter electrode can flow saline for before the hydrogel gets saturated. Cation exchange membranes,, may be a preferred choice for use as an ionically conductive membrane that separates the hydrogel from the electrolytic fluid. While these membranes have limitations such as allowing proton diffusion (that affects electrolyte flow requirements) and slow water diffusion, these limitations have been further accounted for with design features of the electrode that will be further discussed.

For the specific embodiments herein described the choice of a cation membrane for use as the ionic conducting membrane layer 34 create certain limitations, as discussed above. These limitations can be ameliorated by design considerations of the at least one hydrogel layer 38, 42 that interfaces with the ionically conductive membrane layer 34 forming the ionically conducting assembly 32. The electrode according to the present invention can have at least one, but may have as many as five (5) hydrogel layers disposed between the ionically conductive membrane layer 34 and the skin surface (not shown) of the patient. As noted in the embodiments of FIGS. 1 and 2, two hydrogel layers 38 and 42 are provided, though an electrode design based on a single hydrogel layer can also be advantageous. It is to be noted that certain hydrogel characteristics are modifiable and advantageous effects can be achieved by optionally having different hydrogel layers laminated together. Parameters that should be considered in the electrode design include levels of ingredients that may affect conductivity, pH, and mechanical properties. As seen in FIG. 1, the inner hydrogel layer 38 interfaces the ionically conductive membrane layer 34 to the outer hydrogel layer 42, with the outer hydrogel layer 42 then interfacing directly with the skin surface (not shown) of the patient about which the electrode 10 is disposed.

According to this embodiment, the inner and outer hydrogel layers 38, 42 exhibit a buffering system similar to that described in WO2021/178040A1, herein incorporated by reference in its entirety, in which the buffering compound(s) present in the hydrogel layer(s) bind with any hydrogen ions that have diffused therethrough and neutralizes them. Accordingly, the pH at the patient skin interface cannot begin to decrease until the buffering capacity of the hydrogel has first been fully depleted, thus adding yet another layer of patient safety to permit extended use of the herein described electrode. In at least one embodiment, the initial pH of the buffered hydrogel is 7, but this parameter can be modified to range from being mildly acidic (pH=about 5) to fully basic (pH=12). In at least one embodiment, the buffering compound used in the hydrogel layers 38, 42 is magnesium acetate, although it will be understood that other suitable buffering compounds can be selected for use.

In addition and with the introduction of the flowing electrolytic fluid and ionically conductive membrane layers 34 according to the present electrode design, there are additional design considerations in order to combat any slow diffusion of water through the ionically conductive membrane layer 34. One such consideration is the crosslink density of the hydrogel. Polymer meshes or matrices are a component of nearly every hydrogel. These meshes are designed to trap water therein and allow the hydrogel to be in a solid form. Hydrogels are produced when an uncured liquid gel is exposed to irradiation that links polymer chains together, thus creating a polymer matrix that stiffens the gel. To that end, an inverse relationship exists between the crosslink density and both gel adhesiveness and ability to absorb water into its body. Therefore, a gel exposed to higher irradiation during manufacturing will become less adhesive, but more resistant to mechanical changes from water. Knowing this, the inner hydrogel layer 38 may contain a higher level of crosslink density than that of the outer hydrogel layer 42. By having the inner hydrogel layer 38 at a higher crosslink density, water diffusion through the ionically conducting membrane layer 34 will be slowed due to the resistance to mechanically swelling of a highly linked hydrogel. This creates two (2) factors that slows water transport to the skin-interfacing (outer) hydrogel layer 42, one factor being the ionically conductive membrane layer 34 and the second factor being the highly cross linked hydrogel. The loss of adhesion in the inner hydrogel layer 38 will be less impactful to usability of the electrode 10 than that of the skin-interfacing outer hydrogel layer 42 because the inner hydrogel layer 38 may optionally have smaller size dimensions that would allow the outer hydrogel layer 42 to seal both the ionically conductive membrane layer 34 and the inner hydrogel layer 38 to the insulating layer 26 at the outer borders or edges.

Another challenge that exists relates to optimizing the flow and electrolyte parameters in order to effectively extend the treatment lifespan while also maintaining patient usability of the electrode. In the present embodiment, cation exchange membranes are used as the material for the ionically conductive membrane layer 34, which can therefore readily allow for protons (units of acid) to diffuse through into the skin interfacing outer hydrogel layer 42, electrolyte flow must be optimized to dispose of these protons before they can reach the ionically conductive membrane layer 34. This optimization can be done through a balance of two (2) key parameters; namely, proton neutralizing compounds and electrolyte flow rate. First, the effect of proton neutralizing compounds within the electrolytic fluid should be considered. Similar to the buffering compound within the hydrogel layer(s), the flowing electrolytic fluid also may contain compounds that neutralize protons. Neutralizing agents for this purpose may include chemical buffers, hydroxide salts or a combination of each. Both of these types of neutralizing agents bind to protons and neutralize the acid, but rely on different mechanisms. More specifically, buffers are aqueous solutions consisting of a mixture of a weak acid and its conjugate base. In the presence of excess hydrogen ions in solution, the conjugate base will protonate the hydrogen ion to become a weak acid. Alternatively and if the solution is too basic, the weak acid will deprotonate its proton to form water and its conjugate base, thus keeping the pH within a range specific to that compound. In a preferred embodiment, the buffering compound used in the flowing electrolytic fluid is potassium bicarbonate, although it will be understood that other buffering compounds can be selected.

The other neutralizing agent, namely hydroxide salts, are compounds that dissociate into hydroxide ions and a cation. The ratio of hydroxide ions to protons in solution is what literally drives the pH value of the solution. With high amounts of hydroxide ions dissolved in the flowing electrolyte, any protons produced at the anode will combine with free hydroxides to form water. Although both agents are applicable for neutralizing protons, use of a buffering agent may be more desirable for reasons related to safety. This safety issue relates to how the capacity of these agents affects the pH level of the flowing electrolyte. It is beneficial to add as much buffer or hydroxide salt as possible (which is limited by their solubility) in order to maximize electrode lifespan. In the case of hydroxide ions, dissolving as much of the hydroxide salts as possible would lead to an undesirably high pH fluid. If the electrode were to become damaged and leak, this fluid could cause major irritation or damage to the skin. On the other hand, buffers can be loaded to capacity, maintain a neutral pH, and still have the same neutralizing capability as a hydroxide salt. It is understood that while buffering compounds, hydroxide salts or a combination of each can be used, buffering compounds are preferred.

Other significant parameters of the flowing electrolytic fluid that optimizes treatment lifespan and patient usability are the reservoir volume and flow rate. To better understand the reservoir system as a whole, reference is made to FIG. 3(a), which schematically depicts a patient limb 104 (partially shown) with a metallic implant 108 embedded with the joint space. A fully assembled fluid dual channel fluid exchange electrode 212, as per FIGS. 4(a) and 4(b) is demonstrated by way of example. The electrode 212 is adhered to the limb 104 as a counter electrode of a CVCES-based treatment system, the electrode 212 being adhered according to this example to the leg of the patient and which is configured for providing DC stimulation, represented by arrows 120 to the implant 108 via coupling of the electrode to a potentiostat 130 having respective communication lines 134 and 136 to the electrical lead 54 and the implant 108. Details relating to an exemplary treatment system are provided in WO2021/146238 A1, which is incorporated by reference in its entirety. The inflow and outflow tubes 228, 232 of the electrode 212 are interfaced with an electrolyte reservoir 140. The electrolytic fluid 144 within the reservoir 140 is facilitated through the electrode 212 via a pump 150. The pump 150 is configured to control the flow rate of the electrolyte fluid 144 with a specified RPM value. As further depicted in FIG. 3(a), treatment lifespan can be monitored, for example, using either coulomb (charge) counting software or firmware that can be provided on the potentiostat 130 or alternatively, this monitoring can be accomplished using an integrated pH sensor 160 in the electrolyte reservoir 140, in which the pH sensor 160 can provide feedback to release more solid neutralizing agent, depicted as 161 into the electrolyte reservoir 140.

It is understood that the reservoir 140 of electrolytic fluid 144 is capable of literally any size and therefore the reservoir 140 could theoretically hold an infinite volume of electrolyte fluid 144. This is an ideal state because there would then be an infinite supply of neutralizing agent and the electrode lifespan could therefore be infinite in terms of preventing acid buildup in the membrane and hydrogel layers. However, an infinite, or even a very large volume reservoir is not desired or practicable from a patient usability standpoint. Moreover, an ideal state from a usability standpoint would be to have as little reservoir volume as possible (less mobility restriction, the device would be lighter, etc.). Accordingly, the reservoir 140 has been optimized so that in one preferred embodiment, the reservoir volume contains between about 100 mL and 1 L of electrolytic fluid 144. However, it is understood that the volume of the reservoir 130 can functionally vary anywhere from about 10 mL to 1000 L of electrolytic fluid.

When using an electrolytic fluid containing a hydroxide salt (as opposed to a buffer), the contents of the reservoir 140 can be facilitated to the electrode by the pump 150, programmed to operate at a specified flow rate. In one preferred embodiment, the flow rate of the pump 150 can be dictated by the level of current being controlled by the potentiostat 130. The foregoing control is important because the amount of current generated through the electrode/treatment system dictates the number of protons being generated within the fluid exchange electrode 212. If there is an inappropriate or insufficient amount of new electrolyte fluid being pumped into the electrode 212 (i.e., either too much or too little fluid), then the electrolyte fluid volume within the electrode body may either become too alkaline or too acidic because the rate of proton creation fails to match the rate of proton neutralization. Having the electrolyte fluid become too acidic creates a risk factor for the skin of the patient as well as electrode damage whereas having electrolyte fluid within the electrode that is too alkaline is only a risk if the electrode becomes damaged and leaks. Therefore, if the pump 150 can constantly or at least periodically update its flow rate to match the creation of new protons (dictated by the current), then the pH within the fluid exchange electrode 212 can effectively be maintained at an appropriate level. The communication of measured current to the pump 150 is facilitated through a separate communication line 154. Alternatively and if the electrolytic fluid 144 uses a buffering solution, then the pump 150 can be programmed to operate at a constant flow rate.

FIG. 6 shows experimental data gathered using a benchtop prototype of a version of a fluid exchange electrode detailing at least some of the advantageous effects of the present invention. The prototype electrode used in the experiments contained an orthopedic implant cathode chamber that was separated from the fluid exchange electrode by 3% agar with 0.9% NaCl, which is representative of human tissue in terms of mechanical and electrical values. Hydrogel and ionically conducting membrane layers were applied to the agar surface as they would be found in a clinical setting. Behind the membrane layers, an enclosed flow cell or chamber was established used to analyze how current from the anode interacts with the flowing electrolyte. In this flow cell, anodic current, pump flow rate, concentrations of neutralizing agents, and treatment time could be modulated to help understand and optimize the electrode.

Referring to the graph of FIG. 6, five (5) example experiments, or tests, were conducted with results provided at graph 500, in which the pH of the enclosed flow chamber was tracked of the course of 6 hours. In each of these particular experiments, the initial pH value inside the chamber was about 7. At time 0, a DC stimulation of 200 mA was turned on (for all depicted experiments) and an externally coupled pump was turned on to the RPM value denoted by the graphs key. More specifically, the following RPM values were used: 50 rpm, 5 rpm, 15 rpm, 10 rpm and 10 rpm, which are represented by trendlines 504, 508, 512, 516 and 520, respectively. In the case of every test with the exception of the trendline 520 (10 rpm buffered), the inflowing electrolyte was at a pH of 12. As gleaned from FIG. 6, and at higher RPM values (higher flow rate) such as 50 rpm and 15 rpm, see trendlines 504, 512, the influx of pH 12 electrolytic fluid overpowers the production of protons at the anode and the overall volume becomes alkaline, creating a hazard if the electrode was to be punctured or otherwise caused to leak.

Additionally, it can be seen that at 5 rpm the proton production overpowered the slow influx of basic electrolyte and became too acidic, which could diffuse through the membrane layers and eventually reach the skin surface. At an RPM of 10, see trendline 516, the PH balance rises initially but then begins to decrease towards acidic levels. At about time 150 min, a boost of solid NaOH was added to the fill chamber, causing a pH increase back to 10 as the NaOH dissolved before the pH falls again in a similar pattern. The addition of the NaOH boost represents how the chamber pH can be kept within acceptable bounds by monitoring the solution pH, such as using a pH sensor and control system having software that can subsequently add dissolvable neutralizing agent. This feature can be built into the electrode system to be automatic. As to the remaining test, see trendline 520, both the chamber electrolyte and inflowing electrolyte were buffered with potassium bicarbonate, which clearly holds the pH steady at around 8 throughout the entire test. This trendline illustrates the advantage of using a buffer. However, at some extrapolated time point, the buffer will eventually reach its capacity and pH will then begin to decrease. After analyzing this data, it is clear that flow rate is more important when using hydroxide salts, and less important when using buffer. In the case of buffer, as long as the flow rate of the pump is not exceptionally slow to the point where the internal volume of the electrode begins to exceed its buffer capacity faster than new buffer can inflow, then the pH will remain steady until the buffer capacity of the entire volume (reservoir+electrode) is exceeded.

Another negative aspect of prior electrode designs that are used for cathodic DC voltage treatments of implant biofilms is that they inadequately relieve thermal increases of the skin, particularly at higher treatment levels. In addition to each of the previously stated advantages, the herein described design provides a superior mechanism for cooling thermal increases at the skin to electrode interface. The tissue between the implant and the electrode body that the treatment current flows through is thought to heat according to traditional power laws in which circuit components with higher resistances will give off greater thermal losses. The skin, having a larger resistance as compared to muscle or other body fluid, is thought to experience higher temperature rise in the presence of high treatment current. This has been evident in tests run with prior generations of electrodes with no fluid flow component, as measured with temperature probes in different parts of the tissue. The herein described design having a constantly circulating volume of electrolyte fluid has been shown to substantially increase cooling efficiency of the skin over prior generations under the same test parameters. The mechanism behind the increased cooling efficiency is thought to be the fact that the heat of the skin is now allowed to sink into a large volume of fluid, and not just the thin hydrogel provided between the stimulation surface and the skin of prior known designs. Also, because the fluid is constantly flowing to and from the reservoir, the heat sink volume is limited to that inside the electrode compartment, but extends to the total volume between the electrode and the reservoir.

Further design improvements on the electrode's system, and more specifically the electrolyte reservoir, can enhance the already improved thermal regulation properties of the fluid exchange electrode design. Thermal regulation of the skin can improve even further if the fluid volume is chilled to lower temperatures. To do this and according to at least one version shown in FIG. 3(b), with similar parts being labeled with the same reference numbers for clarity, the system may incorporate a cooling element 170 in the electrolyte reservoir 140 that can control the fluid temperature. This cooling element 170 may exist in multiple forms including, but not limited to a refrigeration unit that employs compressors or chemicals, or a thermoelectric device. Ideally, the fluid should be as cold as possible (without freezing) to achieve maximum results. However, and depending on how much treatment current is running through the tissue, maximum cooling may not always be necessary and could simply be a waste of power. For this latter reason, the system may further employ a temperature sensor 174 and a software feedback mechanism 178, shown schematically, which may include a controller wherein the software feedback mechanism 178 can actively take temperature measurements of the system and control the cooling element 170, as needed, to keep the skin at a desired temperature. The temperature sensor 174 may be preferably located in the skin-hydrogel interface, or alternatively be disposed either inside the body of the electrode 212, or in the electrolyte reservoir 140. The foregoing arrangement of thermal regulation features can be separately disposed as shown in the system of FIG. 3(b), or included in the system configuration of FIG. 3(a).

Using the electrode design of FIG. 1 as an example and in terms of electrode manufacture, the first step in assembling this design is to attach the metallic backing layer 18 and more specifically the bottom surface thereof to the upper surface of the conducting electrode surface layer 14. To do this, the materials must be cut and measured to the specific size configuration and placed in a way that there is an even border of the conducting electrode surface layer 14 extending past the periphery of the metallic backing layer 18 on all sides, often referred to as “island placement”. As previously discussed, recessing the metallic backing layer 18 in this manner prevents electrolyte fluid 14, FIG. 2, from contacting the metallic backing layer 18, which is usually made from copper or silver, and which could cause the metallic backing layer 18 to corrode. The metallic backing layer 18 can be attached to the conducting electrode surface layer 18 using a strong conductive adhesive that can be evenly spread.

The next step is to connect the electrical lead 54 to the metallic backing layer 18 in a way that ensures electrical contact with the surface of the metallic backing layer 18. Again, the metallic backing layer 18 must be sealed in a way that limits its contact with the passing electrolyte fluid 80, FIG. 2. To tend to this issue, a water-resistant adhesive can be used on the extended border of the conducting electrode surface layer 14 and sealed to the “ceiling” of insulative backing 22 with the metallic backing layer 18 being layered in between. After aligning the inflow and outflow tubes 46, 50 in the correct orientation according to the followed configuration, the edge or border 58 can be sealed with the exception of an opening for the mechanical support mesh layer 30 to be inserted. Alternatively and if the configuration chosen is the dual channel electrode 212 featured in FIGS. 4(a) and 4(b), the center seal should not be done at this stage. These layers can preferably be attached using a heat source. The mechanical support mesh layer 30 can then be placed in the opening formed between the conducting electrode surface layer 14 and the insulating layer 26. The remaining opening is then sealed and closed. During this latter step, one should ensure that the connection of the end of the electrical lead 54 is outside of the sealed layers. Setting aside the partially assembled unit and according to these specific designs, the ionically conductive membrane layer 34 is then sealed to the inner hydrogel layer 38 ensuring that there is an even extended border of the hydrogel layer 38 remaining. No additional adhesive is needed when attaching these two layers. The outer hydrogel layer 42 is then aligned and attached to the inner hydrogel layer 38. As previously noted, the hydrogel “layer” can be a single layer having one hydrogel or alternatively include a plurality of hydrogel portions as described in this embodiment, or can be separate and distinct hydrogel layers. Lastly, the partially assembled unit is attached to the hydrogel assembly. An adhesive is applied that is compatible with the insulating layer 26, the adhesive being applied to the border of the inner hydrogel layer 38 and sealing the border of the inner hydrogel layer 38 to the insulating layer 26 to complete the ionically conducting assembly 32. As assembled, the ionically conductive membrane layer 34 is beneath and placed in direct contact with the insulating layer 26.

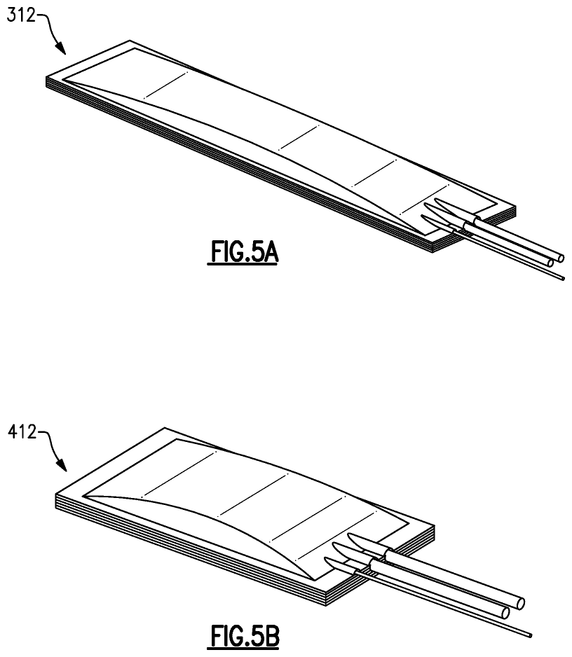

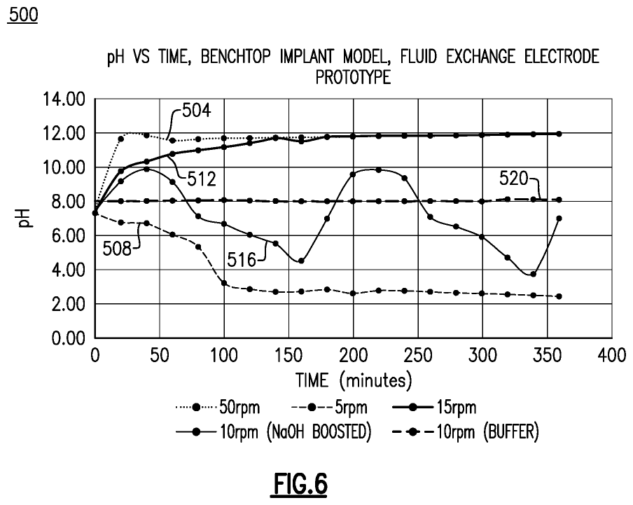

As noted, a configuration for a fluid exchange electrode 10 is featured in FIG. 1 that defines a straight or linear flow to a defined flow chamber within the electrode body via the inflow and outflow tubes 46, 50. Alternatively, FIGS. 4(a) and 4(b) depict a configuration having parallel flow channels defined within the electrode body, wherein the structures are fluidically sealed on each of the four sides of the insulating layers 22, 26, FIG. 1 and which can be done using simple tooling. However, it will be understood that these configurations can be easily transformed into a longer device, such as depicted as electrodes 312 and 412, see FIGS. 5(a) and 5(b). These configurations have the same assembly process and layered structure as electrode 212, with electrode 312 having a longer length dimension in order to allow for more surface area contact with the patient's skin and electrode 412 having a larger width dimension than that of electrode 212. Having the ability to manufacture fluid exchange electrodes to different lengths and widths allows the electrode to adhere circumferentially around patient limbs of different sizes, or limb circumferences. By extending the electrode around a majority of the patient's limb circumference creates an evenly distributed therapy on the implant itself. However, unlike the prior art described in Applicant's WO 2021/178040A1, the fluid exchange electrode does not depend on large surface area of the stimulating surface to extend the electrodes lifespan because it has the ability to neutralize and dispel acidic buildup regardless of surface area size. For this reason, the herein described fluid exchange electrode does not need to necessarily be a fully circumferential wrap. That is, the fluid exchange electrode could be made as multiple smaller electrode shapes or nodes that could be placed more strategically around the limb or implant to promote even therapy and optimize space.

While the invention has been described in terms of particular variations and illustrative figures, those of ordinary skill in the art will recognize that the invention is not limited to the variations or figures described. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well.

To the extent that the claims recite the phrase “at least one of” in reference to a plurality of elements, this is intended to mean at least one or more of the listed elements, and is not limited to at least one of each element. For example, “at least one of an element A, element B, and element C,” is intended to indicate element A alone, or element B alone, or element C alone, or any combination thereof. “At least one of element A, element B, and element C” is not intended to be limited to at least one of an element A, at least one of an element B, and at least one of an element C.

The terminology used herein is for the purpose of describing particular embodiments only and is not intended to be limiting. As used herein, the singular forms “a,” “an,” and “the” are intended to include the plural forms as well, unless the context clearly indicates otherwise. It will be further understood that the terms “comprise” (and any form of comprise, such as “comprises” and “comprising”), “have” (and any form of have, such as “has” and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The corresponding structures, materials, acts, and equivalents of all means or step plus function elements in the claims below, if any, are intended to include any structure, material, or act for performing the function in combination with other claimed elements as specifically claimed. The description set forth herein has been presented for purposes of illustration and description, but is not intended to be exhaustive or limited to the form disclosed. Many modifications and variations will be apparent to those of ordinary skill in the art without departing from the scope and spirit of the disclosure. The embodiment was chosen and described in order to best explain the principles of one or more aspects set forth herein and the practical application, and to enable others of ordinary skill in the art to understand one or more aspects as described herein for various embodiments with various modifications as are suited to the particular use contemplated and in accordance with the following appended claims. Additional embodiments include any one of the embodiments described above and described in any and all exhibits and other materials submitted herewith, where one or more of its components, functionalities or structures is interchanged with, replaced by or augmented by one or more of the components, functionalities or structures of a different embodiment described above.

### PARTS LIST OF FIGS. 1-6

10 counter or return (fluid exchange) electrode
14 conducting electrode surface layer
18 metallic backing layer
22 insulative backing
26 insulation layer
30 mesh support layer
32 ionically conducting assembly
34 ionically conductive membrane layer
38 inner hydrogel layer
42 outer hydrogel layer
46 inflow tube
50 outflow tube
54 electrical lead
58 edge seal(s)
62 interior (open) volume
66 pattern of conduction perforations
70 arrow
80 electrolytic or electrolyte fluid
84 arrow
88 ports
104 leg, patient
108 implant
120 arrows, charge/current
130 potentiostat
134 line
136 line
140 electrolyte reservoir
144 volume of electrolyte or electrolytic fluid
150 pump
154 line
160 pH sensor
161 neutralizing agent
164 buffer
170 cooling element
174 temperature sensor
178 software feedback mechanism
212 fluid exchange electrode
216 conductive electrode material layer
220 metallic backing layer
224 insulative backing
226 open chamber
227 indented portion
228 insulating layer
229 inflow tube
232 outflow tube
240 mesh support layer
312 electrode
412 electrode
500 graph
504 trendline
508 trendline
512 trendline
516 trendline
520 trendline

This detailed description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims. As noted, it will be understood that other suitable variations and modifications will be readily apparent and understood by those of sufficient skill in the field reading the preceding detailed description and as will further be understood from the following listed claims.

1. A fluid exchange electrode comprising:
an electrode body;
a conductive electrode material layer disposed within the electrode body;
an ionically conductive assembly configured to interface with the skin surface of a patient; and
an inflow tube and an outflow tube coupled to an interior of the electrode body, wherein the inflow tube is configured for connection to a source of electrolytic fluid to permit a volume of electrolytic fluid to flow through the electrode body, wherein the insulation layer includes a plurality of spaced conduction perforations and in which the ionically conductive assembly is disposed beneath the defined flow chamber.

2. The electrode of claim 1, wherein the conductive electrode material layer is formed from at least one from the group consisting of carbon vinyl, platinum and rhodium.

3. The electrode of claim 1, further comprising an insulation layer and an insulative covering that combine to define a flow chamber within the electrode body to which the inflow and outflow tubes are attached and wherein the conductive electrode material layer is disposed within the flow chamber.

4. (canceled)

5. The electrode of claim 3, further comprising a support layer disposed between the conductive electrode material layer and the insulation layer.

6. The electrode of claim 4, in which the inflow tube and the outflow tube are disposed on opposing sides of the electrode body.

7. The electrode of claim 4, in which the inflow tube and the outflow tube are each disposed in parallel relation on one side of the electrode.

8. (canceled)

9. The electrode of claim 1, wherein the ionically conducting assembly comprises an ionically conductive membrane layer disposed between one or more hydrogel layers and the insulation layer, wherein the conduction perforations of the ionically conducting membrane layer permits ionic conduction to the membrane layer.

10. The electrode of claim 9, wherein the ionically conductive membrane layer is made from at least one of the group consisting of a cation exchange membrane, an anion exchange membrane or a solid-state electrolyte.

11. The electrode of claim 10, wherein the ionically conductive membrane layer is made from a cation exchange membrane.

12. The electrode of claim 3, further comprising a metallic backing layer disposed between the conductive electrode material layer and the insulative covering.

13. The electrode of claim 12, in which an extending lead is attached to one of the metallic backing layer and the conductive electrode material layer, the extending lead being configured for attachment to a voltage supply.

14. The electrode of claim 9, wherein the one or more hydrogel layers are linked to the ionically conductive membrane layer.

15. The electrode of claim 14, wherein at least one of the one or more hydrogels is buffered.

16. The electrode of claim 15, in which the buffer is magnesium acetate.

17. The electrode of claim 15, in which there are two or more hydrogels in which the hydrogel linked to the ionically conductive membrane layer has a higher crosslink density than the hydrogel configured for contact with the skin surface of the patient.

18. A system for treatment of metal implants for the removal of bacteria, the system comprising:
a device capable of producing a DC voltage;
a working electrode coupled to the device capable of producing a DC voltage, the working electrode being the metal implant;
a counter electrode coupled to the device capable of producing a DC voltage, the counter electrode comprising a multi-layered electrode body having:
a conductive electrode material layer disposed in a flow chamber of the electrode; and
an ionically conducting assembly configured to directly contact the skin of a patient; and
a source of electrolytic fluid coupled to the counter electrode and configured to permit a volume of electrolytic fluid to flow through the electrode body.

19. The system of claim 18, further comprising a pump coupled to the source of electrolytic fluid and configured to facilitate flow to the counter electrode.

20. The system of claim 19, in which the counter electrode includes an inflow tube and an outflow or return tube, each coupled to the source of electrolytic fluid.

21. The system of claim 19, in which the counter electrode includes an inflow tube and an outflow tube in which the inflow tube is connected to the source of electrolytic fluid and the outflow tube is connected to a waste receptacle.

22. The system of claim 18, in which the electrolytic fluid includes at least one dissolved salt.

23. The system of claim 22, in which the at least one dissolved salt is sodium chloride.

24. The system of claim 22, wherein the electrolytic fluid further includes at least one neutralizing agent.

25. The system of claim 21, wherein the at least one neutralizing agent is at least one of a hydroxide salt and a buffer.

26. The system of claim 21, wherein the electrolytic fluid is cooled to enhance thermal regulation of the skin of the patient to which the electrode is attached.

27. The system of claim 26, in which the electrolytic fluid is cooled to temperatures between 30 and 98 degrees Fahrenheit.

28. The system of claim 18, wherein the electrolytic fluid is a viscous gel.

29. The system of claim 18, in which the conductive electrode material layer is made from one of the group consisting of carbon vinyl, platinum and rhodium.

30. The system of claim 29, wherein the flow chamber is defined by an insulation layer and an insulative covering sealed together.

31. The system of claim 30, wherein the counter electrode further comprises a support sheet disposed in the flow chamber to prevent collapse of the flow chamber.

32. The system of claim 18, further comprising an electrical lead coupling the counter electrode with the device capable of producing a DC voltage, the electrical lead being attached to either the conductive surface electrode layer or to a metallic backing sheet disposed in contact with the conductive surface electrode layer.

33. The system of claim 30, wherein the insulation layer includes a plurality of conduction perforations, in which the ionically conducting assembly of the counter electrode comprises an ionically conductive membrane layer attached to the insulation layer and at least one hydrogel layer.

34. The system of claim 33, wherein the at least one hydrogel layer is linked to the ionically conductive membrane layer.

35. The system of claim 34, wherein the ionically conductive assembly is made from one of the group consisting of a cation exchange membrane, an anion exchange membrane or a solid-state electrolyte.

36. The system of claim 35, wherein the ionically conductive membrane layer is made from a cation exchange membrane.

37. The system of claim 19, wherein the source of electrolytic fluid is a reservoir.

38. The system of claim 37, wherein the reservoir retains about 1 mL to 1000 L of electrolytic fluid.

39. The system of claim 38, wherein the reservoir retains about 100 mL to 1 L of electrolytic fluid.

40. The system of claim 37, including a pH sensor disposed in the reservoir and configured to detect changes in neutralizing agent.

41. The system of claim 40, wherein the electrolytic fluid includes at least one of a buffer and a hydroxide salt, and in which the system is configured to automatically add neutralizing agent to the reservoir based on changes detected by the pH sensor or indicating a change for exchange of electrolytic fluid in the reservoir.

42. The system of claim 37, wherein the fluid reservoir contains a cooling element configured to actively cool the electrolytic fluid to order to maintain and regulate the temperature of the electrolytic fluid in order to optimize thermal relief of the skin of the patient.

43. The system of claim 42, in which the cooling element is part of a sealed system that uses a chemical refrigerant.

44. The system of claim 42, in which the cooling element is a thermoelectric cooling device.

45. The system of claim 42, further comprising a temperature sensor disposed in the reservoir, the temperature sensor being configured to detect changes in the temperature of the electrolytic fluid in the reservoir.

46. The system of claim 43, including a closed loop feedback mechanism having the temperature sensor and a controller coupled to the temperature sensor, the controller being configured to control the chemical refrigerant in order to regulate the temperature of the electrolytic fluid.

47. The system of claim 37, wherein the device configured for applying a DC voltage is further configured to monitor charge through the counter electrode.

48. The system of claim 47, further comprising control logic configured to automatically add neutralizing agent or indicating that an exchange of electrolytic fluid is needed in the reservoir based on monitored charge in the counter electrode.

49. The system of claim 40, wherein the electrolytic fluid is not buffered and includes at least one hydroxide salt, the system further comprising control logic for adjusting the flow rate of the pump based on monitored current from the device capable of producing a DC voltage.

50. A method for increasing the lifespan of an electrode used for biofilm removal treatment of implants by cathodic DC current, the method comprising:
coupling a source of electrolytic fluid to the electrode; and
causing electrolyte fluid to be circulated into the electrode during treatment.
wherein the electrode includes an electrode body having an interior flow chamber disposed between a pair of insulation layers, and an electrically conductive electrode surface layer disposed within the defined flow chamber, the method further comprising:
providing a plurality of conduction perforations in one of the insulation layers; and
providing an ionically conducting assembly beneath the insulation layer having the plurality of perforations and a skin surface of a patient.

51. The method of claim 50, in which the ionically conducting assembly includes an ionically conducting membrane layer and one or more hydrogel layers, in which a said hydrogel layer is configured to interface directly with the skin surface of the patient.

52. The method of claim 51, wherein the ionically conductive membrane layer is made from one of the group consisting of a solid state electrolyte, a cation exchange membrane and an anion exchange membrane.

53. The method of claim 52, wherein the ionically conductive membrane layer is made from a cation exchange layer.

54. The method of claim 53, wherein the electrolytic fluid contains at least one dissolved salt.

55. The method of claim 54, wherein the at least one dissolved salt is sodium chloride.

56. The method of claim 54, wherein the electrolytic fluid further contains at least one neutralizing agent.

57. The method of claim 56, wherein the at least one neutralizing agent is at least one of a hydroxide salt and a buffer.

58. The method of claim 50, wherein electrolytic fluid is directed to flow through the electrode with used electrolytic fluid being recirculated to the source of electrolytic fluid.

59. The method of claim 50, wherein electrolytic fluid is directed to flow through the electrode with used electrolytic fluid being directed to a waste receptacle.
